(19) United States
(12) Patent Application Publication
(10) Pub. No.: US 2025/0040842 A1
(43) Pub. Date: Feb. 6, 2025

(54) APPARATUS AND METHOD FOR UTILIZING MONOCHROME LIGHT TO DETERMINE BLOOD OXYGEN SATURATION IN A NON-INVASIVE MANNER

(71) Applicant: STMicroelectronics International N.V., Geneva (CH)

(72) Inventors: Nicola PICOZZI, Nettuno (IT); Alessandro GUMIERO, Milan (IT); Marco ODONI, Cinisello Balsamo (IT); Luigi DELLA TORRE, Lissone (IT); Daniele PAPARELLO, Fondi (IT)

(21) Appl. No.: 18/364,275

(22) Filed: Aug. 2, 2023

(51) Int. Cl. A61B 5/1455; A61B 5/00

(52) U.S. Cl. CPC A61B 5/14551; A61B 5/742

(57) ABSTRACT

An example method, apparatus, and pulse oximeter device for determining a blood oxygen saturation value in a subject are provided. The example apparatus may include a light source configured to emit monochrome light of a single emitted wavelength. The light source may be positioned to direct the monochrome light at a portion of a subject. The example apparatus may further include a light sensing diode configured to receive an unabsorbed portion of the monochrome light. The apparatus may also include a controller configured to determine a blood oxygen saturation value based on one or more characteristics of the unabsorbed portion of the monochrome light comprising light of the single emitted wavelength.

### TECHNOLOGICAL FIELD

Embodiments of the present disclosure relate generally to determining blood oxygen saturation in a subject, and more particularly, utilizing monochrome light to determine blood oxygen saturation in a subject in a non-invasive manner.

### BACKGROUND

Many non-invasive vital sign monitoring applications utilize photoplethysmography (PPG) techniques to determine vital signs. PPG generally involves illuminating the skin or tissue of a subject with a light source and then detecting changes in the unabsorbed light with a photodiode. As the volume of blood in the vessels changes due to the blood being pumped through them during the cardiac cycle, the amount of light absorbed by the subject's tissue also changes. In addition, certain proteins in the subject's blood may absorb certain wavelengths of light more readily than other proteins. Thus, certain blood measurements may be determined based on the unabsorbed light.

Applicant has identified many technical challenges and difficulties associated with determining blood oxygen saturation values in a non-invasive manner. Through applied effort, ingenuity, and innovation, Applicant has solved problems related to the determination of blood oxygen saturation values by developing solutions embodied in the present disclosure, which are described in detail below.

### BRIEF SUMMARY

Various embodiments are directed to an example method, apparatus, and pulse oximeter device for determining a blood oxygen saturation value in a subject. An example apparatus is provided. In some embodiments, the example apparatus comprises a light source configured to emit monochrome light, wherein the light source is positioned to direct the monochrome light at a portion of a subject, and wherein the monochrome light comprises light of a single emitted wavelength. The example apparatus further comprising a light sensing diode configured to receive an unabsorbed portion of the monochrome light; and a controller configured to determine a blood oxygen saturation value based on one or more characteristics of the unabsorbed portion of the monochrome light comprising light of the single emitted wavelength.

In some embodiments, the light sensing diode is positioned adjacent to the light source, and the unabsorbed portion of the monochrome light is reflected by the portion of the subject.

In some embodiments, the light sensing diode is positioned opposite the portion of the subject as the light source, and the unabsorbed portion of the monochrome light traverses the portion of the subject.

In some embodiments, the light sensing diode is a photodiode configured to produce an electrical output based on an intensity of light received comprising the single emitted wavelength.

In some embodiments, the controller is further configured to extract an unabsorbed period of the unabsorbed portion of the monochrome light.

In some embodiments, the unabsorbed period corresponds to a sub-portion of the unabsorbed portion of the monochrome light between two consecutive local minimums.

In some embodiments, the unabsorbed period is transmitted to a blood oxygen saturation machine learning model configured to determine the blood oxygen saturation value based on the unabsorbed period.

In some embodiments, the blood oxygen saturation machine learning model is a blood oxygen saturation neural network.

In some embodiments, the light source is one of a light-emitting diode (LED) and a laser.

In some embodiments, the single emitted wavelength of the monochrome light is between 400 nanometers and 10 micrometers.

A method for determining a blood oxygen saturation value is further provided. In some embodiments, the method comprises causing a light source to emit monochrome light, wherein the monochrome light is directed at a portion of a subject, and wherein the monochrome light comprises light having a single emitted wavelength; receiving an electrical output representing an intensity of an unabsorbed portion of the monochrome light received at a light sensing diode; and determining a blood oxygen saturation value based on one or more characteristics of the unabsorbed portion of the monochrome light comprising light of the single emitted wavelength.

In some embodiments, the light sensing diode is positioned adjacent to the light source, and the unabsorbed portion of the monochrome light is reflected by the portion of the subject.

In some embodiments, the light sensing diode is positioned opposite the portion of the subject as the light source, and the unabsorbed portion of the monochrome light traverses the portion of the subject.

In some embodiments, the light sensing diode is a photodiode configured to produce an electrical output based on an intensity of light received comprising the single emitted wavelength.

In some embodiments, the method further comprises extracting an unabsorbed period of the unabsorbed portion of the monochrome light.

In some embodiments, the unabsorbed period corresponds to a sub-portion of the unabsorbed portion of the monochrome light between two consecutive local minimums.

In some embodiments, the method further comprises transmitting the unabsorbed period to a blood oxygen saturation machine learning model, and receiving from the blood oxygen saturation machine learning model the blood oxygen saturation value, wherein the blood oxygen saturation value is based on the unabsorbed period.

In some embodiments, the blood oxygen saturation machine learning model is a blood oxygen saturation neural network.

A pulse oximeter device is further provided. In some embodiments, the pulse oximeter device comprises a housing. The housing comprising an upper portion comprising a light source, and a lower portion comprising a light sensing diode. In some embodiments, a portion of a subject is received into a space defined between the upper portion and the lower portion. In addition, the light source is configure to emit monochrome light directed toward the lower portion. Also, the monochrome light comprises light having a single emitted wavelength. Further, the light sensing diode is configured to receive an unabsorbed portion of the monochrome light. The pulse oximeter device may further include a controller configured to determine a blood oxygen saturation value based on one or more characteristics of the unabsorbed portion of the monochrome light.

In some embodiments, the pulse oximeter device may further include an electronic display, wherein the electronic display is configured to display the blood oxygen saturation value.

### BRIEF DESCRIPTION OF THE DRAWINGS

Reference will now be made to the accompanying drawings. The components illustrated in the figures may or may not be present in certain embodiments described herein. Some embodiments may include fewer (or more) components than those shown in the figures in accordance with an example embodiment of the present disclosure.

FIG. 1 illustrates an example process for determining a blood oxygen saturation value in a subject based on two PPG signals resulting from two light sources emitting two different wavelengths of light.

FIG. 2 illustrates a block diagram of an example blood oxygen saturation measurement device in accordance with an example embodiment of the present disclosure.

FIG. 3 depicts a block diagram of example components of an example controller in accordance with one or more embodiments of the present disclosure.

FIG. 4 illustrates an example process for determining a blood oxygen saturation value in a subject using monochrome light in accordance with an example embodiment of the present disclosure.

FIG. 5 illustrates an example blood oxygen saturation machine learning model for determining a blood oxygen saturation value in accordance with an example embodiment of the present disclosure.

FIG. 6 illustrates an example flow chart depicting an example process for determining a blood oxygen saturation value in a subject using monochrome light in accordance with an example embodiment of the present disclosure.

FIG. 7 illustrates an example pulse oximeter device utilizing monochrome light to determine a blood oxygen saturation value in accordance with an example embodiment of the present disclosure.

### DETAILED DESCRIPTION

Example embodiments will be described more fully hereinafter with reference to the accompanying drawings, in which some, but not all embodiments of the inventions of the disclosure are shown. Indeed, embodiments of the disclosure may be embodied in many different forms and should not be construed as limited to the embodiments set forth herein; rather, these embodiments are provided so that this disclosure will satisfy applicable legal requirements. Like numbers refer to like elements throughout.

Various example embodiments address technical problems associated with determining a blood oxygen saturation value in a subject or patient. As understood by those of skill in the field to which the present disclosure pertains, there are numerous example scenarios in which a person may desire to determine the blood oxygen saturation value in a subject.

For example, monitoring vital parameters, such as heart rate, heart rate variability, blood pressure, and blood oxygen saturation (SpO₂) in a non-invasive manner has become an important part of everyday health routines. In particular, blood oxygen saturation may indicate the correct functioning of the systems that are responsible for delivering oxygen to the blood and removing carbon dioxide from the blood. These systems may include the functioning of the heart and the overall health of the circulatory system, the functioning of the lungs and the overall health of the respiratory system, and so on. In addition, the blood oxygen saturation value may provide indications regarding the environment of a subject, for example, if an environment lacks sufficient oxygen. As such, many electronic devices such as watches, phones, fitness trackers, and tablets now support monitoring of blood oxygen saturation in some capacity.

Many non-invasive methods for measuring blood oxygen saturation utilize photoplethysmography (PPG) techniques to determine vital signs. PPG generally involves illuminating the skin or tissue of a subject with a light source and then detecting changes in the light with a photodiode. When the light source is directed at the skin or tissue of the subject, depending on the wavelength of light, a portion of the light is reflected by the tissue, a portion of the light is absorbed by the tissue, and a portion of the light passes through the tissue. Two primary PPG techniques include reflectance PPG and transmittance PPG. Reflectance PPG involves capturing and analyzing the light that reflects from the tissue. Transmittance PPG involves capturing and analyzing the light the passes through the tissue. Either one, or both, techniques may be utilized to determine the blood oxygen saturation level in the tissue.

As the volume of blood in the blood vessels changes due to blood being pumped through them, so does the amount of light absorbed by, reflected by, and/or passing through the tissue. Many vital signs measurements may be made leveraging these properties. For example, changes in the light received at a photodiode may be used to indicate the heart rate and heart rate variability of the individual based on the photodiode readings. Similarly, the oxygen saturation of the blood affects the amount and color of light absorbed by the tissue. Photodiode readings may indicate the blood oxygen saturation value of a subject based on the transmitted and/or reflected light (e.g., unabsorbed light).

One of the most difficult problems for manufacturers of mobile devices is to add functionality, such as vital signs monitoring, without significantly changing the size, weight, and power consumption of the mobile device. For example, manufacturers seek to avoid increasing the physical size of the device when providing additional functionality. In addition, manufacturers seek to avoid significant increases in power consumption when adding new device functionality.

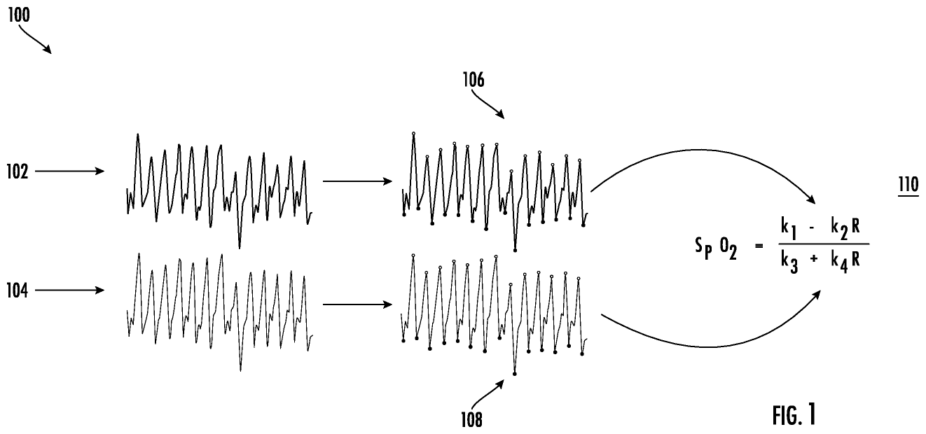

As depicted in the process 100 of FIG. 1, one approach to determining the blood oxygen saturation value in a non-invasive manner is to use two light sources with two different wavelengths, for example, a first light source emitting red light (e.g., light having a wavelength between 620 and 720 nanometers) and a second light source emitting infrared light (e.g., light having a wavelength between 720 nanometers and 2520 nanometers). In general, the proteins within the blood of a subject react differently to light of different wavelengths, including oxygenated hemoglobin and non-oxygenated hemoglobin.

Thus, as shown in FIG. 1, two different PPG signals are generated, a first PPG signal 102 corresponding with the unabsorbed light from the first light source and a second PPG signal 104 corresponding with the unabsorbed light from the second light source. Based on the first PPG signal 102, one or more first PPG periods 106 may be recorded. Similarly, based on the second PPG signal 104, one or more second PPG periods 108 may be recorded. Utilizing the blood oxygen saturation equation 110, a blood oxygen saturation may be determined based on the one or more first PPG periods 106 corresponding to the first light source and the one or more second PPG periods 108 corresponding to the second light source.

As shown in FIG. 1, the value R in the blood oxygen saturation equation 110 includes a ratio of values derived from the first light source having a first wavelength divided by a ratio of values derived from the second light source having a second wavelength. As such, a plurality of light sources emitting light having at least two different wavelengths are necessary. The corrective coefficients (k₁-k₄) in the blood oxygen saturation equation 110 are estimated constants observed during the calibration phase of a device, often determined during a clinical trial supervised by medical staff.

A plurality of light sources exhibit a number of problems for manufacturers of mobile devices, such as phones, watches, bands, and the like. A plurality of light sources may occupy limited space within a mobile device, in some cases, requiring a change to the form factor of the mobile device to house a second light source. In addition, a plurality of light sources consumes additional power, reducing the battery life of mobile devices utilizing battery power.

The various example embodiments described herein utilize various techniques to determine the blood oxygen saturation in a subject utilizing a single light source emitting monochrome light. For example, in some embodiments, a light source is configured to emit monochrome light toward a portion of a subject. As described herein, a portion of the light is absorbed in the tissue of the subject, based on the contents of the blood within the tissue of the subject. The unabsorbed portion of the emitted monochrome light is received by a light sensing diode. In some embodiments, the unabsorbed portion is reflected by the portion of the subject and received by a light sensing diode proximate the light source. In some embodiments, the unabsorbed portion is transmitted through the portion of the subject and received by a light sensing diode opposite the light source. Based on the characteristics of the unabsorbed light, the blood oxygen saturation of the subject may be determined.

In some embodiments, one or more periods of the received unabsorbed portion of the transmitted monochrome light may be extracted. The one or more periods may be transmitted to a machine learning model trained to determine the blood oxygen saturation measurement based on the one or more periods of the unabsorbed light.

As a result of the herein described example embodiments and in some examples, the size and power consumption of a non-invasive blood oxygen saturation measurement device may be greatly reduced. In addition, the accuracy of the blood oxygen saturation measurement device may satisfy associated medical standards.

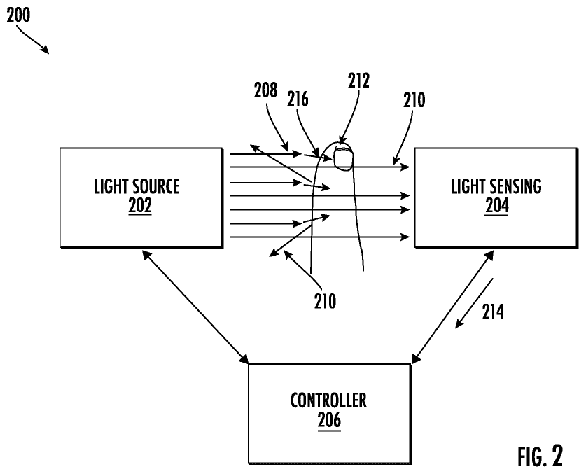

Referring now to FIG. 2, an example blood oxygen saturation measurement device 200 is provided. As depicted in FIG. 2, the example blood oxygen saturation measurement device 200 includes a light source 202 configured to emit monochrome light 208 toward a portion of a subject (e.g., finger of a subject 212). An unabsorbed portion 210 of the emitted monochrome light 208 passes through the finger of the subject 212 and is received by a light sensing diode 204. As further depicted in FIG. 2, both the light source 202 and the light sensing diode 204 are communicatively connected to a controller 206.

As depicted in FIG. 2, the example blood oxygen saturation measurement device 200 includes a light source 202 configured to emit monochrome light 208. A light source 202 may be any device, bulb, semiconductor, diode, laser, or other photon-emitting structure configured to generate monochrome light 208 and positioned to direct the light toward a finger of a subject 212. The light source 202 is configured to generate monochrome light 208 at a specific wavelength (e.g., single emitted wavelength). The blood oxygen saturation measurement device 200 system may operate with any wavelength of monochrome light 208 emitted from the light source 202 capable of extracting information from the blood volume (e.g., red, green, blue, infrared, ultraviolet, etc.). As a non-limiting example, the light source 202 may be configured to generate infrared monochrome light 208 having a wavelength between 750 nanometers and 10 micrometers. Further non-limiting examples may include red monochrome light 208 having a wavelength between 620 nanometers and 750 nanometers; green monochrome light 208 having a wavelength between 520 nanometers and 570 nanometers; and other wavelengths of monochrome light 208 from the visible (e.g., 400 nanometers to 750 nanometers) and non-visible spectrum (e.g., 750 nanometers to 10 micrometers). In some embodiments, the light source 202 may comprise an array of light-emitting diodes (LEDs), each generating monochrome light 208 at the same specified wavelength.

As further depicted in FIG. 2, the monochrome light 208 of the example blood oxygen saturation measurement device 200 is directed toward a finger of a subject 212 (e.g., portion of a subject). Although depicted as a finger in FIG. 2, the finger of the subject 212 may be any skin, tissue, or other blood carrying portion of the subject's body. In some embodiments, the portion of the subject may enable the passage of light from one side of the body, through the skin and/or tissue, and to the other side of the portion of the subject. For example, such portions may include a finger (e.g., finger of the subject 212), a toe, or an earlobe.

In an instance in which the monochrome light 208 encounters the finger of the subject 212, a sub-portion of the monochrome light 208 is absorbed by the finger of the subject 212 (e.g., absorbed portion 216). Various structures of the finger of the subject 212 may absorb portions of the monochrome light 208 based on the wavelength of the light and the particular structure. For example, oxygenated blood comprising oxygenated hemoglobin absorbs more red monochrome light 208, while deoxygenated blood allows more red monochrome light 208 to pass through.

As depicted in FIG. 2, the unabsorbed portion 210 of the monochrome light 208 is received by a light sensing diode 204. The unabsorbed portion 210 is any portion of the monochrome light 208 that is not absorbed by the finger of the subject 212. In some embodiments, for example when utilizing reflectance PPG techniques, the unabsorbed portion 210 of the monochrome light 208 reflects off the finger of the subject 212 and is received by a light sensing diode 204 placed adjacent to the light source 202. In some embodiments, for example when utilizing transmission PPG techniques (as depicted in FIG. 2), the unabsorbed portion 210 of the monochrome light 208 is the portion of the monochrome light 208 that passes through the finger of the subject 212. In such an embodiment, the unabsorbed portion 210 may be received by a light sensing diode 204 positioned in the line of sight of the light source 202, however, on the opposite side of the finger of the subject 212 from the light source 202.

As further depicted in FIG. 2, the example blood oxygen saturation measurement device includes a light sensing diode 204. A light sensing diode 204 may be any set of one or more integrated circuits, devices, sensors, photodiodes, or other structures that produce an electric current proportional to the intensity of light received at the light sensing diode 204. For example, the electric current output by the light sensing diode 204 may increase as the number of photons that strike the light sensing diode 204 per second increases. In such an embodiment, the electric current output from the light sensing diode 204 may be used to determine the intensity of light striking the light sensing diode 204. In some embodiments, the light sensing diode 204 may be a light sensitive semiconductor diode that creates an electron-hole pair at the p-n junction when a photon of sufficient energy strikes the light sensing diode 204.

A light sensing diode 204 is positioned to receive at least a sub-portion of the unabsorbed portion 210 of the monochrome light 208. For example, in one embodiment, the light source 202 is positioned to direct monochrome light 208 toward the finger of the subject 212 and the light sensing diode 204 is disposed within the line of sight of the light source 202 on the opposite side of the finger of the subject 212. Thus, monochrome light 208 received at the light sensing diode 204 from the light source 202 is the unabsorbed portion 210 of monochrome light 208 that passes through the finger of the subject 212. In another embodiment, the light sensing diode 204 is disposed adjacent to the light source 202 on the same side of the finger of the subject 212 as the light source 202. In such an embodiment, monochrome light 208 received at the light sensing diode 204 from the light source 202 is the unabsorbed portion 210 of monochrome light 208 that reflects off the finger of the subject 212.

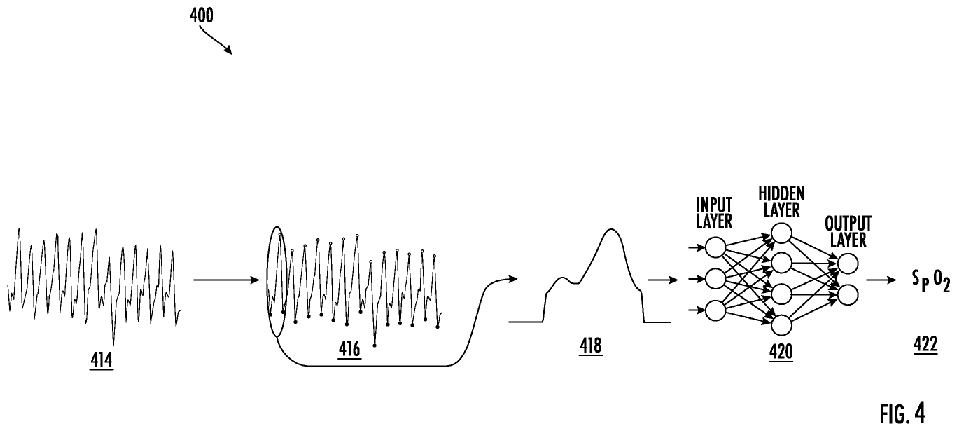

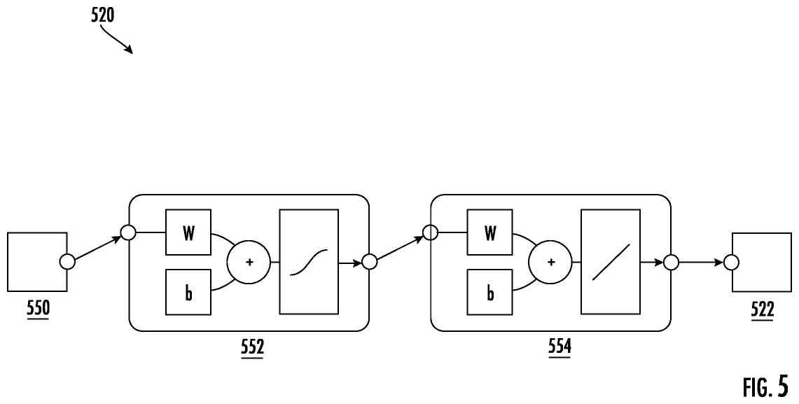

A light sensing diode 204 is further configured to transmit the electrical current representing the intensity of light received at the light sensing diode 204 (e.g. PPG signal 214) to the controller 206. The PPG signal 214 is any electronic signal representing the unabsorbed portion 210 of the emitted monochrome light 208 received at the light sensing diode 204. As further described in relation to FIG. 4, the controller 206 may perform operations to determine the blood oxygen saturation value based on features of the PPG signal 214. For example, the controller 206 may extract one or more periods of the PPG signal for further analysis. As further described in relation to FIG. 4-FIG. 5, the controller 206 may implement a machine learning model configured to determine the blood oxygen saturation value based on the extracted features of the PPG signal 214.

In some embodiments, the light sensing diode 204 may be mounted and/or electrically connected to a structure, such as a printed circuit board, configured to position the one or more light sensing diodes 204 to receive the unabsorbed portion 210 of the monochrome light 208 and provide circuitry to generate the PPG signal 214 for transmission to the controller 206 and various components of the blood oxygen saturation measurement device 200 (e.g., controller 206).

As further depicted in FIG. 2, the example blood oxygen saturation measurement device 200 includes a controller 206. A controller 206 is any computing device electrically connected to the light source 202 and the light sensing diode 204 and configured to exchange control/status messages with the light source 202 and the light sensing diode 204.

Control/status messages exchanged with the light source 202 may include electrical signals configured to control the frequency and/or wavelength of the monochrome light 208 generated by the light source 202. Command messages may further control the shape and/or pattern of the output monochrome light 208.

Command/status messages exchanged with the light sensing diode 204 may include the receipt of the PPG signal 214 used to determine a blood oxygen saturation value. In addition, the controller 206 may be configured to control parameters of the light sensing diode 204, such as the sampling rate, the spectral responsivity, the response time, and other similar parameters of the light sensing diode 204.

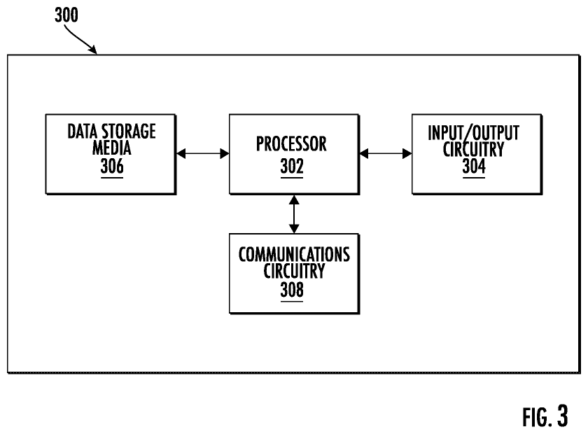

A controller 206 may be embodied by one or more computing system such as apparatus 300 shown in FIG. 3. The apparatus 300 may include processor 302, data storage media 306, input/output circuitry 304, and a communications circuitry 308. Although these components 302-308 are described with respect to functional limitations, it should be understood that the particular implementations necessarily include the use of particular hardware. It should also be understood that certain of these components 302-308 may include similar or common hardware. For example, two sets of circuitries may both leverage use of the same processor, network interface, storage medium, or the like to perform their associated functions, such that duplicate hardware is not required for each set of circuitries.

In some embodiments, the processor 302 (and/or co-processor or any other processing circuitry assisting or otherwise associated with the processor) may be in communication with the data storage media 306 via a bus for passing information among components of the apparatus. The data storage media 306 is non-transitory and may include, for example, one or more volatile and/or non-volatile memories. In other words, for example, the data storage media 306 may be an electronic storage device (e.g., a computer-readable storage medium). The data storage media 306 may include one or more databases. Furthermore, the data storage media 306 may be configured to store information, data, content, applications, instructions, or the like for enabling the apparatus 300 to carry out various functions in accordance with example embodiments of the present invention.

The processor 302 may be embodied in a number of different ways and may, for example, include one or more processing devices configured to perform independently. In some preferred and non-limiting embodiments, the processor 302 may include one or more processors configured in tandem via a bus to enable independent execution of instructions, pipelining, and/or multithreading. The use of the term “processing circuitry” may be understood to include a single core processor, a multi-core processor, multiple processors internal to the apparatus, and/or remote or “cloud” processors.

In some preferred and non-limiting embodiments, the processor 302 may be configured to execute instructions stored in the data storage media 306 or otherwise accessible to the processor 302. In some preferred and non-limiting embodiments, the processor 302 may be configured to execute hard-coded functionalities. As such, whether configured by hardware or software methods, or by a combination thereof, the processor 302 may represent an entity (e.g., physically embodied in circuitry) capable of performing operations according to an embodiment of the present invention while configured accordingly. Alternatively, as another example, when the processor 302 is embodied as an executor of software instructions (e.g., computer program instructions), the instructions may specifically configure the processor 302 to perform the algorithms and/or operations described herein when the instructions are executed.

In some embodiments, the apparatus 300 may include input/output circuitry 304 that may, in turn, be in communication with processor 302 to provide output to the user and, in some embodiments, to receive an indication of a user input. The input/output circuitry 304 may comprise a user interface and may include a display, and may comprise a web user interface, a mobile application, a query-initiating computing device, a kiosk, or the like.

In embodiments in which the apparatus 300 is embodied by a limited interaction device, the input/output circuitry 304 includes a touch screen and does not include, or at least does not operatively engage (i.e., when configured in a table mode), other input accessories such as tactile keyboards, track pads, mice, etc. In other embodiments in which the apparatus is embodied by a non-limited interaction device, the input/output circuitry 304 may include at least one of a tactile keyboard (e.g., also referred to herein as keypad), a mouse, a joystick, a touch screen, touch areas, soft keys, and other input/output mechanisms. The processor and/or user interface circuitry comprising the processor may be configured to control one or more functions of one or more user interface elements through computer program instructions (e.g., software and/or firmware) stored on a memory accessible to the processor (e.g., data storage media 306, and/or the like).

The communications circuitry 308 may be any means such as a device or circuitry embodied in either hardware or a combination of hardware and software that is configured to receive and/or transmit data from/to a network and/or any other device, circuitry, or module in communication with the apparatus 300. In this regard, the communications circuitry 308 may include, for example, a network interface for enabling communications with a wired or wireless communication network. For example, the communications circuitry 308 may include one or more network interface cards, antennae, buses, switches, routers, modems, and supporting hardware and/or software, or any other device suitable for enabling communications via a network. Additionally, or alternatively, the communications circuitry 308 may include the circuitry for interacting with the antenna/antennae to cause transmission of signals via the antenna/antennae or to handle receipt of signals received via the antenna/antennae.

It is also noted that all or some of the information discussed herein can be based on data that is received, generated and/or maintained by one or more components of apparatus 300. In some embodiments, one or more external systems (such as a remote cloud computing and/or data storage system) may also be leveraged to provide at least some of the functionality discussed herein.

Referring now to FIG. 4, an example process 400 for determining a blood oxygen saturation value 422 based on a PPG signal is provided. As depicted in FIG. 4, the process 400 includes receiving a PPG signal at stage 414, characteristic extraction at stage 416, data preparation at stage 418, and machine learning processing at stage 420 configured to output the blood oxygen saturation value 422.

As depicted in FIG. 4, the example process 400 includes receiving a PPG signal at stage 414. A PPG signal is any electronic signal representing the light received at a light sensing diode (e.g., light sensing diode 204) positioned to capture the unabsorbed portion (e.g., unabsorbed portion 210) of monochrome light (e.g., monochrome light 208) after encountering a portion of a subject (e.g., finger of subject 212). In some embodiments, the PPG signal may be generated based on the unabsorbed portion of the monochrome light that is reflected by the subject when encountering the subject's tissue and received at the light sensing diode. In some embodiments, the PPG signal may be generated based on the unabsorbed portion of the monochrome light that is transmitted through the subject without being absorbed by the subject's tissue and received at the light sensing diode.

Certain determinations about the subject may be made based on the PPG signal. Structures within the tissue of the subject, particularly structures within the blood of the subject, absorb the transmitted monochrome light. Thus, as the volume of the blood in the tissue increases, the amount of light absorbed increases. The volume of blood in the tissue is used to determine certain vital signs, such as heart rate and heart rate variability.

In addition, the blood of a subject contains certain proteins such as hemoglobin. Hemoglobin is a protein in the red blood cells of a subject that carries oxygen through the body. Hemoglobin picks up oxygen at the lungs and releases the oxygen to various tissues of the body. In general, oxygenated hemoglobin (or hemoglobin carrying oxygen) absorbs light differently than de-oxygenated hemoglobin (or hemoglobin not carrying oxygen). For example, oxygenated blood comprising oxygenated hemoglobin absorbs more red monochrome light when compared to deoxygenated blood comprising primarily deoxygenated hemoglobin.

As further depicted in FIG. 4, the process 400 includes characteristic extraction at stage 416. Characteristic extraction includes any detection and extraction of features and/or characteristics of the PPG signal received at stage 414. One such characteristic may be the period of the PPG signal (e.g., unabsorbed period). The period of the PPG signal comprises a segment of the PPG signal representing one complete cycle. For example, a period of the PPG signal may include the segment of the PPG signal between two consecutive troughs (or local minimums) in the PPG signal, as depicted in FIG. 4. Extracting the period from the PPG signal may include identifying distinct points in the PPG signal, such as local minimums or local maximums (or crests), and sampling the PPG signal between two consecutive distinct points. Sampling the PPG signal may include reading a value associated with the PPG signal at a sampling rate. For example, a sampling rate may be determined based on a total number of samples desired, or based on a time between successive samples.

Additional characteristics of the PPG signal extracted at stage 416 may include the wavelength of the PPG signal, the amplitude of the PPG signal, and/or absolute and relative location of events identified in the PPG signal. For example, certain phases of the heart operation may be determined from a PPG signal, such as the systole phase corresponding to heart contraction and the diastole phase corresponding to heart relaxation. Other characteristics of the PPG signal may include the location of the dicrotic notch, the difference between the intensity of light received at the local maximum and the local minimum, the pulsatile component (AC) and baseline component (DC) of one or more periods in the PPG signal, and so on.

As further depicted in FIG. 4, the process 400 includes a data preparation stage 418. At the data preparation stage 418, the extracted characteristics are prepared for consumption by a machine learning model. Certain data preparation techniques may be applied to the extracted characteristics. For example, as depicted in FIG. 4, one such extracted characteristic may be an extracted period from the PPG signal. The extracted period may be resampled, normalized, padded, or otherwise modified in preparation for transmission to a machine learning model for further processing. Further data preparation may include purging the received PPG signal of outlier data or other noise.

The data preparation stage 418 may further include vectorization of the extracted characteristics. For example, a vector may be created wherein each entry in the vector corresponds with a data point of extracted data. Data points may be the sampled values of the PPG signal period (e.g., unabsorbed period), the calculated characteristics of the PPG signal, or any other extracted characteristic determined at stage 416.

As further depicted in FIG. 4, the process 400 includes a machine learning processing stage 420. The machine learning processing stage 420 may comprise hardware, software, firmware, and/or specifically configured circuitry to train and execute a machine learning model capable of receiving inputs related to the extracted characteristics of a PPG signal and outputting a blood oxygen saturation value based on the determined outcome of the machine learning model.

Machine learning is a method used to devise complex models and algorithms that lend themselves to prediction. A machine learning model is a computer-implemented algorithm that may learn from data with or without relying on rules-based programming. These models enable reliable, repeatable decisions and results and uncovering of hidden insights through machine-based learning from historical relationships and trends in the data. In some embodiments, the machine learning model is a clustering model, a regression model, a neural network, a random forest, a decision tree model, a classification model, or the like.

A machine learning model is initially fit or trained on a training dataset (e.g., a set of examples used to fit the parameters of the model). The model may be trained on the training dataset using supervised or unsupervised learning. The model is run with the training dataset and produces a result, which is then compared with a target, for each input vector in the training dataset. Based on the result of the comparison and the specific learning algorithm being used, the parameters of the model are adjusted. In some embodiments, the model can be trained and/or trained in real-time (e.g., online training) while in use.

The machine learning model may be any suitable model for determining the blood oxygen saturation value 422 from the extracted characteristics. For example, the machine learning model may be some form of neural network. An example neural network machine learning model is described in relation to FIG. 5. The underlying machine learning models may be learning models (supervised or unsupervised). As examples, such algorithms may be prediction (e.g., linear regression) algorithms, classification (e.g., decision trees, k-nearest neighbors) algorithms, time-series forecasting (e.g., regression-based) algorithms, association algorithms, clustering algorithms (e.g., K-means clustering, Gaussian mixture models, DBscan), Bayesian methods (e.g., Naïve Bayes, Bayesian model averaging, Bayesian adaptive trials), image to image models, (e.g., FCN, PSPNet, U-Net) sequence to sequence models (e.g., RNNs, LSTMs, BERT, Autoencoders) or Generative models (e.g., GANs).

Alternatively, machine learning models may implement statistical algorithms, such as dimensionality reduction, hypothesis testing, one-way analysis of variance (ANOVA) testing, principal component analysis, conjoint analysis, neural networks, support vector machines, decision trees (including random forest methods), ensemble methods, and other techniques. Other ML models may be generative models (such as Generative Adversarial Networks or autoencoders).

In various embodiments, the machine learning models may undergo a training or learning phase before they are released into a production or runtime phase or may begin operation with models from existing systems or models. During a training or learning phase, the machine learning models may be tuned to focus on specific variables, to reduce error margins, or to otherwise optimize their performance. The machine learning models may initially receive input from a wide variety of data, such as the extracted characteristics as described herein.

As further depicted in FIG. 4, the machine learning models of the machine learning processing stage 420 output a blood oxygen saturation value (SₚO₂) 422. The blood oxygen saturation value 422 represents the percentage of oxygen present in a subject's blood. Low blood oxygen saturation values 422 may lead to serious health conditions, including damage to individual organ systems, such as the brain or heart. In addition, low blood oxygen saturation values 422 may be an indicator of improper functioning of the respiratory and/or circulatory system of a subject. Thus, real-time measurement of a subject's blood oxygen saturation value 422 may be an important tool in monitoring the health of a subject.

Referring now to FIG. 5, an example embodiment of a blood oxygen saturation machine learning model 520 (e.g., blood oxygen saturation neural network) is provided. As depicted in FIG. 5, the example blood oxygen saturation machine learning model 520 comprises a neural network. The neural network includes an input layer 550, a hidden layer 552, an output layer 554, and an blood oxygen saturation value 522. The blood oxygen saturation machine learning model 520 may be utilized to determine a blood oxygen saturation value (e.g., blood oxygen saturation value 422) based on one or more extracted characteristics of a received PPG signal (e.g., PPG Signal 214). For example, the blood oxygen saturation machine learning model 520 may be leveraged as part of the machine learning processing stage (e.g., machine learning processing stage 420).

As depicted in FIG. 5, the example blood oxygen saturation machine learning model 520 includes an input layer 550. The input layer 550 is configured with a number of neurons, nodes, or input points equal to the number of data points of extracted data comprised in the vector generated during the data preparation stage 418. For example, a vector may be generated based on sampled values from one period of the received PPG signal. Thus, in an instance in which 80 samples are extracted from the PPG signal and a vector comprising the 80 samples is generated, the input layer 550 comprise 80 neurons, each configured to receive a sample of the period of the PPG signal (e.g., unabsorbed period).

As further depicted in FIG. 5, the blood oxygen saturation machine learning model 520 comprises a hidden layer 552. A hidden layer 552 is a set of neurons positioned between the input layer 550 and output layer 554 of a neural network. A neuron in the hidden layer 552 comprises a data construct configured to receive an input, apply a weight, add a bias, and direct the weighted, biased input through an activation function. The weights and bias of a neuron within the hidden layer may be automatically and/or manually adjusted based on training data and/or during classification of extracted characteristics of the PPG signal. The hidden layer 552 has no constraint with respect to the number of neurons it can contain. In the depicted embodiment of FIG. 5, a hyperbolic tangent function is used as the activation function of each neuron in the hidden layer 552.

As further depicted in FIG. 5, the blood oxygen saturation machine learning model 520 comprises an output layer 554. The output layer 554 receives the output from each of the neurons in the last layer of the hidden layer 552 and produces a blood oxygen saturation value 522 based on the values provided by the hidden layer. In an instance in which one output value is generated (e.g., blood oxygen saturation value 522), the output layer 554 may comprise a single neuron. In the depicted embodiment of FIG. 5, the output layer 554 of the example blood oxygen saturation machine learning model 520 utilizes a linear activation function so as not to alter the values generated at the output layer 554.

As further depicted in FIG. 5, a blood oxygen saturation value 522 is generated based on the extracted characteristics provided by the input layer 550. In some embodiments, the blood oxygen saturation value 522 may comply with pertinent medical standards. For example, in some embodiments, medical standards require a blood oxygen saturation value 522 to be within 2 percent of the actual blood oxygen saturation value of the subject. Various parameters within the blood oxygen saturation machine learning model 520 may be updated to improve the accuracy of the generated blood oxygen saturation value 522. For example, the architecture of the hidden layer 552 to include more layers and/or a reconfiguration of the neurons within the hidden layer 552. In addition, the vector provided to the input layer 550 may include more and/or different extracted characteristics from the received PPG signal.

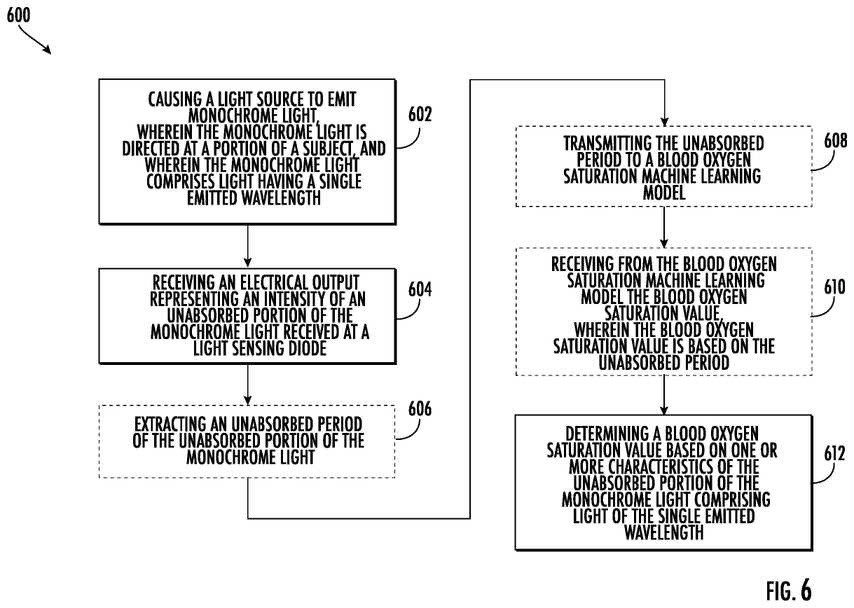

Referring now to FIG. 6, a flowchart illustrating a process 600 for determining a blood oxygen saturation value (e.g., blood oxygen saturation value 422, 522) in a non-invasive manner, is provided. At block 602, the controller (e.g., controller 206) causes a light source (e.g., light source 202) to emit monochrome light (e.g., monochrome light 208), wherein the monochrome light is directed at a portion of a subject (e.g., finger of subject 212), and wherein the monochrome light comprises light having a single emitted wavelength. As described herein, the controller may be configured to transmit control and configuration message to the light source, causing the light source to emit monochrome light. In some embodiments, the controller may configure the wavelength, frequency, and/or pattern of the emitted monochrome light. In some embodiments, the controller may configure the light source to emit light automatically at a pre-determined rate.

At block 604, the controller receives an electrical output (e.g., PPG signal 214) representing an intensity of an unabsorbed portion (e.g., unabsorbed portion 210) of the monochrome light received at a light sensing diode (e.g., light sensing diode 204). As described herein, the electrical output generated by the light sensing diode (or PPG signal) results from the portion of the monochrome light that is not absorbed in the tissue of the subject. In some embodiments, the PPG signal may result from the light reflected off the subject. In some embodiments, the PPG signal may be based on the monochrome light passing through the subject. The PPG signal includes data related to the blood and the composition of the blood in the portion of the subject.

At block 606, the controller extracts an unabsorbed period of the unabsorbed portion of the monochrome light. As described herein, various characteristics may be determined based on the received PPG signal. For example, characteristics extracted from the PPG signal may include samples extracted from one or more periods of the PPG signal (e.g., unabsorbed period), the average wavelength of the PPG signal, the average amplitude of the PPG signal, and/or the absolute and/or relative location of events identified in the PPG signal. Example events may include certain phases of the operation of the heart determined from a PPG signal, such as the systole phase corresponding to heart contraction and the diastole phase corresponding to heart relaxation. Other characteristics of the PPG signal may include the location of the dicrotic notch, the difference between the intensity of light received at the local maximum and the local minimum, the pulsatile component (AC) and baseline component (DC) of one or more periods in the PPG signal, and so on.

In some embodiments, an extracted, unabsorbed period of the PPG signal may be sampled at discrete intervals to generate a vector representing the PPG signal value at sampled intervals over a full period.

At block 608, the controller transmits the unabsorbed period to a blood oxygen saturation machine learning model (e.g., blood oxygen saturation machine learning model 520). As described in relation to FIG. 4-FIG. 5, in some embodiments, a machine learning model may be designed and trained to determine a blood oxygen saturation value based on one or more extracted characteristics of the PPG signal, such as the unabsorbed period. The one or more extracted characteristics may be formatted into a vector as dictated by the machine learning model and transmitted to the machine learning model for further processing.

At block 610, the controller receives from the blood oxygen saturation machine learning model the blood oxygen saturation value, wherein the blood oxygen saturation value is based on the unabsorbed period. As described herein, the machine learning model may generate a blood oxygen saturation value based on the extracted period of the PPG signal. The extracted period comprises a number of features and characteristics that are representative of the blood oxygen saturation of the subject encountered by the monochrome light. For example, the PPG signal may indicate features such as certain phases of the heart operation, the location of the dicrotic notch, the difference between the intensity of light received at the local maximum and the local minimum, the pulsatile component (AC) and baseline component (DC) of the extracted period of the PPG signal, and so on. The various weights and biases within the hidden layer of the machine learning model may utilize these and other characteristics of the period of the PPG signal to determine a blood oxygen saturation value.

At block 612, the controller determines a blood oxygen saturation value (e.g., blood oxygen saturation value 422, 522) based on one or more characteristics of the unabsorbed portion of the monochrome light comprising light of the single emitted wavelength. As described in relation to block 610, in some embodiments, the blood oxygen saturation value is determined by a machine learning model.

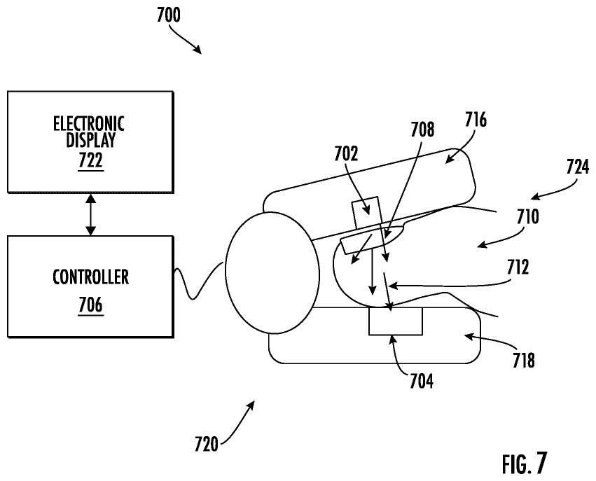

Referring now to FIG. 7, an example pulse oximeter device 700 is provided. As depicted in FIG. 7, the example pulse oximeter device 700 comprises a housing 720 having an upper portion 716 and a lower portion 718. The upper portion 716 comprises an LED 702 (e.g., light source) configured to emit monochrome light 708 toward the lower portion 718 of the housing 720. A space 724 is defined between the upper portion 716 and the lower portion 718, such that a portion of a subject 710 may be received between the upper portion 716 and the lower portion 718. The lower portion 718 comprises a photodiode 704 (e.g., light sensing diode) positioned to receive the unabsorbed portion 712 of the monochrome light 708 passing through the portion of the subject 710. As further depicted in FIG. 7, a controller 706 is electrically connected to the LED 702 and the photodiode 704. The controller 706 is further electrically connected to an electronic display 722.

As depicted in FIG. 7, the example pulse oximeter device 700 utilizes a single LED 702 and transmission mode PPG techniques to generate a PPG signal based on the unabsorbed portion 712 of the monochrome light 708 passing through the portion of the subject 710 and received at the photodiode 704. In some embodiments, the controller 706 may be disposed within the housing 720 of the pulse oximeter device 700. Further, in some embodiments, the controller 706 may comprise a machine learning model (e.g., blood oxygen saturation machine learning model 520) to determine the blood oxygen saturation value of the subject based on the transmission of monochrome light 708 from a single LED 702. Utilizing a single LED 702 reduces the power consumption of the pulse oximeter device 700 and reduces the space required compared to a pulse oximeter device using two light sources.

As further depicted in FIG. 7, the pulse oximeter device 700 includes an electronic display 722. The electronic display 722 may be any screen comprising pixels configured to display information related to the operation of the pulse oximeter device 700. In some embodiments, the electronic display may be mounted on a surface of the housing 720.

While this detailed description has set forth some embodiments of the present invention, the appended claims cover other embodiments of the present invention which differ from the described embodiments according to various modifications and improvements. For example, one skilled in the art may recognize that such principles may be applied to any electronic device that utilizes photoplethysmography techniques to determine vital signs of a subject. For example, a smart watch, a health monitoring band, a mobile device, a tablet, or other electronic device configured to determine vital signs in a non-invasive manner.

Within the appended claims, unless the specific term “means for” or “step for” is used within a given claim, it is not intended that the claim be interpreted under 35 U.S.C. 112, paragraph 6.

Use of broader terms such as “comprises,” “includes,” and “having” should be understood to provide support for narrower terms such as “consisting of,” “consisting essentially of,” and “comprised substantially of” Use of the terms “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

1. An apparatus comprising:
a light source configured to emit monochrome light,
wherein the light source is positioned to direct the monochrome light at a portion of a subject, and
wherein the monochrome light comprises light of a single emitted wavelength;
a light sensing diode configured to receive an unabsorbed portion of the monochrome light; and
a controller configured to determine a blood oxygen saturation value based on one or more characteristics of the unabsorbed portion of the monochrome light comprising light of the single emitted wavelength.

2. The apparatus of claim 1, wherein the light sensing diode is positioned adjacent to the light source, and
wherein the unabsorbed portion of the monochrome light is reflected by the portion of the subject.

3. The apparatus of claim 1, wherein the light sensing diode is positioned opposite the portion of the subject as the light source, and
wherein the unabsorbed portion of the monochrome light traverses the portion of the subject.

4. The apparatus of claim 3, wherein the light sensing diode is a photodiode configured to produce an electrical output based on an intensity of light received comprising the single emitted wavelength.

5. The apparatus of claim 1, wherein the controller is further configured to extract an unabsorbed period of the unabsorbed portion of the monochrome light.

6. The apparatus of claim 5, wherein the unabsorbed period corresponds to a sub-portion of the unabsorbed portion of the monochrome light between two consecutive local minimums.

7. The apparatus of claim 5, wherein the unabsorbed period is transmitted to a blood oxygen saturation machine learning model configured to determine the blood oxygen saturation value based on the unabsorbed period.

8. The apparatus of claim 7, wherein the blood oxygen saturation machine learning model is a blood oxygen saturation neural network.

9. The apparatus of claim 1, wherein the light source is one of a light-emitting diode (LED) and a laser.

10. The apparatus of claim 1, wherein the single emitted wavelength of monochrome light is between 400 nanometers and 10 micrometers.

11. A method comprising:
causing a light source to emit monochrome light,
wherein the monochrome light is directed at a portion of a subject, and
wherein the monochrome light comprises light having a single emitted wavelength;
receiving an electrical output representing an intensity of an unabsorbed portion of the monochrome light received at a light sensing diode; and
determining a blood oxygen saturation value based on one or more characteristics of the unabsorbed portion of the monochrome light comprising light of the single emitted wavelength.

12. The method of claim 11, wherein the light sensing diode is positioned adjacent to the light source, and
wherein the unabsorbed portion of the monochrome light is reflected by the portion of the subject.

13. The method of claim 11, wherein the light sensing diode is positioned opposite the portion of the subject as the light source, and
wherein the unabsorbed portion of the monochrome light traverses the portion of the subject.

14. The method of claim 13, wherein the light sensing diode is a photodiode configured to produce an electrical output based on an intensity of light received comprising the single emitted wavelength.

15. The method of claim 11, further comprising:
extracting an unabsorbed period of the unabsorbed portion of the monochrome light.

16. The method of claim 15, wherein the unabsorbed period corresponds to a sub-portion of the unabsorbed portion of the monochrome light between two consecutive local minimums.

17. The method of claim 15, further comprising:
transmitting the unabsorbed period to a blood oxygen saturation machine learning model; and
receiving from the blood oxygen saturation machine learning model the blood oxygen saturation value,
wherein the blood oxygen saturation value is based on the unabsorbed period.

18. The method of claim 17, wherein the blood oxygen saturation machine learning model is a blood oxygen saturation neural network.

19. A pulse oximeter device, comprising:
a housing comprising:
an upper portion comprising a light source; and
a lower portion comprising a light sensing diode;
wherein a portion of a subject is received into a space defined between the upper portion and the lower portion;
wherein the light source is configure to emit monochrome light directed toward the lower portion,
wherein the monochrome light comprises light having a single emitted wavelength, and
wherein the light sensing diode is configured to receive an unabsorbed portion of the monochrome light; and
a controller configured to determine a blood oxygen saturation value based on one or more characteristics of the unabsorbed portion of the monochrome light.

20. The pulse oximeter device of claim 19, further comprising an electronic display, wherein the electronic display is configured to display the blood oxygen saturation value.
